SEQUENCE LISTING (2 sequences)

SEQ ID NO: 1 - protein, 269 aa, Thermomyces lanuginosus [sequence omitted]

SEQ ID NO: 2 - protein, 269 aa, synthetic construct [sequence omitted]

(19) United States
(12) Patent Application Publication
(10) Pub. No.: US 2023/0151288 A1
(43) Pub. Date: May 18, 2023

(54) ENZYMATIC OR NON-ENZYMATIC BIODIESEL POLISHING PROCESS

(71) Applicant: Novozymes A/S, Bagsvaerd (DK)

(72) Inventors: Anders Rancke Madsen, Charlottelund (DK); Per Munk Nielsen, Hilleroed (DK); Hon Seng Yee, Kuala Lumpur (MY); Hans Christian Holm, Hellerup (DK)

(73) Assignee: Novozymes A/S, Bagsvaerd (DK)

(21) Appl. No.: 17/932,160

(22) Filed: Sep. 14, 2022

Related U.S. Application Data
(63) Continuation of application No. 15/766,819, filed on Apr. 8, 2018, now abandoned.

(30) Foreign Application Priority Data
Oct. 9, 2015 (MY) PI2015703622

(51) Int. Cl. C10L 1/18; C12P 7/649

(52) U.S. Cl. CPC C10L 1/1802; C12P 7/649; C12Y 301/01003; C10L 2290/543; C10L 2290/544; C10L 2200/0476

(57) ABSTRACT

The present invention provides a method for reducing the level of free fatty acids in biodiesel/fatty acid alkyl esters. The method comprises providing a composition comprising fatty acid alkyl esters, free fatty acids and/or a fatty acid feedstock, reacting said free fatty acids and/or said fatty acid feedstock with alcohol in the presence of one or more liquid lipolytic enzymes to produce fatty acid alkyl esters; and/or reacting the free fatty acids and/or said fatty acid feedstock with alcohol in the presence of one or more non-enzymatic catalysts to produce fatty acid alkyl esters.

### REFERENCE TO A SEQUENCE LISTING

This application contains a Sequence Listing in computer readable form. The computer readable form is incorporated herein by reference.

### FIELD OF THE INVENTION

The invention provides a process for reducing the level of free fatty acids in biodiesel/fatty acid alkyl esters. The process comprises reducing the amounts of free fatty acids, in the oil phase/light phase by
i) reacting said free fatty acids and/or said fatty acid feedstock with alcohol in the presence of one or more liquid lipolytic enzymes to produce fatty acid alkyl esters; and/or
ii) reacting the free fatty acids and/or said fatty acid feedstock with alcohol in the presence of one or more non-enzymatic catalysts to produce fatty acid alkyl esters.

### BACKGROUND ART

Fatty acid alkyl esters may be used as fuel, biodiesel, in standard diesel engines. Biodiesel can be used alone, or blended with fossil diesel. Biodiesel has become more attractive recently because of its environmental benefits.

Although biodiesel is at present primarily produced chemically (using e.g., NaOH and/or sodium methoxide as catalyst), there are several associated problems to restrict its development, such as pre-processing of oil due to high contents of free fatty acids, need for high alcohol surplus in reaction removal of chemical catalyst from ester and glycerol phase, and removal of inorganic salts during glycerol recovery.

The disadvantages caused by chemical catalysts are largely prevented by using lipolytic enzymes as the catalysts and in recent years interest has developed in the use of lipases in transesterification for the production of biodiesel.

Biodiesel produced by enzymatic bioconversion is, compared with chemical conversion, more environmental friendly. However, with very few exceptions, enzyme technology is not currently used in commercial scale biodiesel production.

Processes for enzymatic production of fatty acid alkyl esters using liquid enzymes are described in e.g., WO 2006/072256, Lv et al. (Process Biochemistry 45 (2010) 446-450) and WO2012/098114.

In processes for production of fatty acid alkyl esters or biodiesel, a fatty acid feedstock is reacted with alcohol, typically methanol, to produce the fatty acid alkyl esters and glycerol. After the fatty acid feedstock has been reacted with alcohol to produce the fatty acid alkyl esters, the oil phase/light phase contains residual free fatty acids. Generally, the presence of free fatty acids is undesirable, and the level of free fatty acids must be reduced to the extent possible: For instance, European standards for biodiesel require that the level of free fatty acids is below 0.25% (w/w). The industrial use of resins for esterification of free fatty acids in glyceride based oils is well-known as pre-treatment step to alkaline chemical biodiesel process. However, esterification of free fatty acids in methyl-esters to FFA levels below 0.25% is very challenging, which is due to a higher water activity as well as the hydroscopic nature of methyl esters. It is also generally recognized that carrying out additional purification or “polishing” of the fatty acid methyl esters in order to remove free fatty acids, causes a loss of fatty acid methyl esters. Hence, there is generally a trade-off between production yield and free fatty acid levels.

Therefore, there is a need for more efficient processes for production of fatty acid alkyl esters or biodiesel, having reduced content of free fatty acids.

### SUMMARY OF THE INVENTION

The invention provides a for reducing the level of free fatty acids in biodiesel/fatty acid alkyl esters, said process comprising
i) providing a composition comprising
   a. an oil phase/light phase that comprises fatty acid alkyl esters, free fatty acids and, optionally, a fatty acid feedstock; and
   b. a water phase/heavy phase that comprises alcohol and water;
ii) reducing the amount of water in said composition, such as reducing the water content of the water phase/heavy phase to be within the range of 0-15% by weight of the water phase/heavy phase and/or reducing the water content of the oil phase/light phase to be within the range of 200-600 ppm;
   and then reducing the amounts of free fatty acids, and optionally the amounts of said fatty acid feedstock in the oil phase/light phase by
iii) reacting said free fatty acids and/or said fatty acid feedstock with alcohol in the presence of one or more liquid lipolytic enzymes to produce fatty acid alkyl esters;
   and/or
iv) reacting the free fatty acids and/or said fatty acid feedstock with alcohol in the presence of one or more non-enzymatic catalysts to produce fatty acid alkyl esters.

### BRIEF DESCRIPTION OF THE FIGURES

FIG. 1: shows a schematic outline of a process for manufacturing fatty acid alkyl esters by enzyme catalysed transesterification of fatty acids or a fatty acid feedstock with alcohol.

FIG. 2 shows four main embodiments of the present invention.

FIGS. 3-9 show results obtained when using methods according to the invention to produce fatty acid methyl esters having a reduced content of free fatty acids.

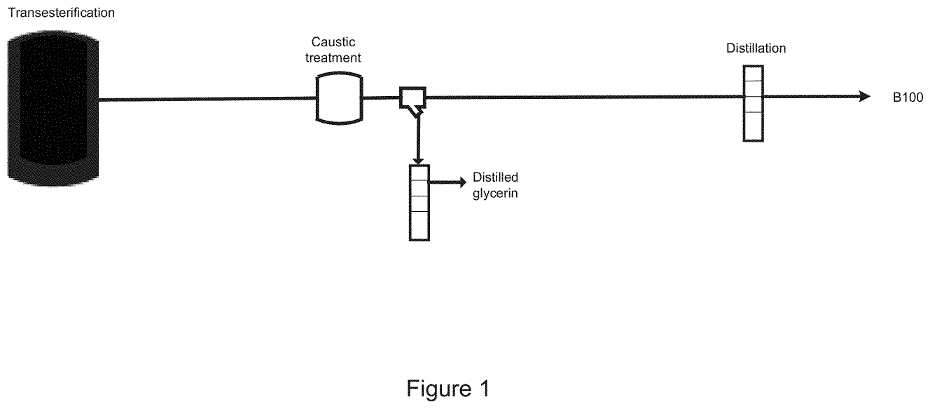

The figure(s) have been included for illustration purposes alone and should in no way be construed as limiting the invention.

### DETAILED DESCRIPTION OF THE INVENTION

### Definitions

Biodiesel: Fatty acid alkyl esters (FAAE) of short-chain alcohols, such as fatty acid methyl esters (FAME) and fatty acid ethyl esters (FAEE) are also called biodiesel, because they are used as an additive to or as replacement of fossil diesel.

Alcohol: The alcohol used in the method of the invention is preferably a short-chain alcohol having 1 to 5 carbon atoms (C₁, C₂, C₃, C₄, or C₅).

Fatty acid feedstock: The term “fatty acid feedstock” is defined herein as a substrate comprising any source of fatty acids, including triglycerides, diglycerides, monoglycerides, or any combination thereof. In principle, any oils and fats of vegetable or animal origin comprising fatty acids may be used as substrate for producing fatty acid alkyl esters in the process of the invention.

### Lipolytic Enzyme

The one or more lipolytic enzyme applied in the method of the present invention is selected from lipases, phospholipases, cutinases, acyltransferases or a mixture of one and more of lipase, phospholipase, cutinase and acyltransferase. The one or more lipolytic enzyme is selected from the enzymes in EC 3.1.1, EC 3.1.4, and EC 2.3. The one or more lipolytic enzyme may also be a mixture of one or more lipases. The one or more lipolytic enzyme may include a lipase and a phospholipase. The one or more lipolytic enzyme includes a lipase of EC 3.1.1.3. The one or more lipolytic enzyme includes a lipase having activity on tri-, di-, and monoglycerides.

Lipases: A suitable lipolytic enzyme may be a polypeptide having lipase activity, e.g., one selected from the Candida antarctica lipase A (CALA) as disclosed in WO 88/02775, the C. antarctica lipase B (CALB) as disclosed in WO 88/02775 and shown in SEQ ID NO:1 of WO2008065060, the Thermomyces lanuginosus (previously Humicola lanuginosus) lipase disclosed in EP 258 068), the Thermomyces lanuginosus variants disclosed in WO 2000/60063 or WO 1995/22615, in particular the lipase shown in positions 1-269 of SEQ ID NO: 2 of WO 95/22615, the Hyphozyma sp. lipase (WO 98/018912), and the Rhizomucor miehei lipase (SEQ ID NO:5 in WO 2004/099400), a lipase from P. alcaligenes or P. pseudoalcaligenes (EP 218 272), P. cepacia (EP 331 376), P. glumae, P. stutzeri (GB 1,372,034), P. fluorescens, Pseudomonas sp. strain SD 705 (WO 95/06720 and WO 96/27002), P. wisconsinensis (WO 96/12012); a Bacillus lipase, e.g., from B. subtilis (Dartois et al. (1993), Biochemica et Biophysica Acta, 1131, 253-360), B. stearothermophilus (JP 64/744992) or B. pumilus (WO 91/16422). Also preferred is a lipase from any of the following organisms: Fusarium oxysporum, Absidia reflexa, Absidia corymbefera, Rhizomucor miehei, Rhizopus delemar (oryzae), Aspergillus niger, Aspergillus tubingensis, Fusarium heterosporum, Aspergillus oryzae, Penicilium camembertii, Aspergillus foetidus, Aspergillus niger, Aspergillus oryzae and Thermomyces lanuginosus, such as a lipase selected from any of SEQ ID NOs: 1 to 15 in WO 2004/099400.

A lipase which is useful in relation to the present invention is a lipase having a sequence identity to the mature polypeptide of SEQ ID NO: 2 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or even 100% sequence identity to the polypeptide shown in positions 1-269 of SEQ ID NO: 2 of WO 95/22615 or to the polypeptide shown in SEQ ID NO:1 of WO2008/065060.

Commercial lipase preparations suitable for use in the process of the invention include LIPOZYME CALB L, LIPOZYME® TL 100L, CALLERA™ TRANS and Eversa® Transform (all available from Novozymes A/S).

Particularly useful lipases may be selected from the group consisting of
(a) a polypeptide comprising or consisting of the amino acid sequence set forth in SEQ ID NO: 1 or SEQ ID NO: 2;
(b) a polypeptide which is a subsequence of the amino acid sequence set forth in SEQ ID NO: 1 or 2;
(c) a polypeptide having at least 60% sequence identity, such as e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to any of the polypeptides defined in (a) and (b).

The lipase set forth in (c) may be a variant the amino acid sequence set forth in SEQ ID NO: 1, wherein the polypeptide comprises the following substitutions T231R and N233R.

The lipase set forth in item (c) may have an amino acid sequence which differs by up to 40 amino acids, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 from the polypeptide of SEQ ID NO: 1 or 2.

The lipase may be a variant of a parent lipase, which variant has lipase activity and has at least 60%, such at least 65%, at least 70%, at least 75%, at least 80%, at least 85%, at least 90%, at least 95% identity, at least 96%, at least 97%, at least 98%, or at least 99%, but less than 100% sequence identity with SEQ ID NO: 1, and comprises substitutions at positions corresponding to T231R+N233R and at least one or more (e.g., several) of D96E, D111A, D254S, G163K, P256T, G91T, G38A, D27R, and N33Q of SEQ ID NO: 1.

In a further embodiment, the lipase is a variant having lipase activity and at least 60% such at least 65%, at least 70%, at least 75%, at least 80%, at least 85%, at least 90%, at least 95% identity, at least 96%, at least 97%, at least 98%, or at least 99%, but less than 100% sequence identity with SEQ ID NO: 1, and comprises substitutions at positions corresponding to T231R+N233R and at least one or more (e.g., several) of D96E, D111A, D254S, G163K, P256T, G91T, G38A, D27R, and N33Q of SEQ ID NO: 1 selected from the group of:
a) D96E T231R N233R;
b) N33Q D96E T231R N233R;
c) N33Q T231R N233R;
d) N33Q D111A T231R N233R;
e) N33Q T231R N233R P256T;
f) N33Q G38A G91T G163K T231R N233R D254S;
g) N33Q G38A G91T D96E D111A G163K T231R N233R D254S P256T;
h) D27R N33Q G38A D96E D111A G163KT231R N233R D254S P256T;
i) D27R N33Q G38A G91T D96E D111A G163KT231R N233R P256T;
j) D27R N33Q G38A G91T D96E D111A G163KT231R N233R D254S;
k) D27R G38A G91T D96E D111A G163K T231R N233R D254S P256T;
l) D96E T231R N233R D254S;
m) T231R N233R D254S P256T;
n) G163K T231R N233R D254S;
o) D27R N33Q G38A G91T D96E G163K T231R N233R D254S P256T;
p) D27R G91T D96E D111A G163KT231R N233R D254S P256T;
q) D96E G163K T231R N233R D254S;
r) D27R G163K T231R N233R D254S;
s) D27R G38A G91T D96E D111A G163K T231R N233R D254S;
t) D27R G38A G91T D96E G163K T231R N233R D254S P256T;
u) D27R G38A D96E D111A G163K T231R N233R D254S P256T:
v) D27R D96E G163K T231R N233R D254S;
w) D27R D96E D111A G163K T231R N233R D254S P256T;
x) D27R G38A D96E G163K T231R N233R D254S P256T.

Such useful variants of a parent lipase are provided, e. g. in WO 2015/049370.

### Lipase Activity:

In the context of the present invention, the lipolytic activity may be determined as lipase units (LU), using tributyrate as substrate. The method is based on the hydrolysis of tributyrin by the enzyme, and the alkali consumption to keep pH constant during hydrolysis is registered as a function of time

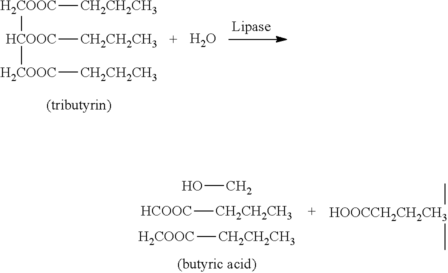

According to the invention, one lipase unit (LU) may be defined as the amount of enzyme which, under standard conditions (i.e. at 30° C.; pH 7.0; with 0.1% (w/v) Gum Arabic as emulsifier and 0.16 M tributyrine as substrate) liberates 1 micromol titrable butyric acid per minute.

Alternatively, lipolytic activity may be determined as Long Chain Lipase Units (LCLU) using substrate pNP-Palmitate (C:16) when incubated at pH 8.0, 30° C., the lipase hydrolyzes the ester bond and releases pNP, which is yellow and can be detected at 405 nm.

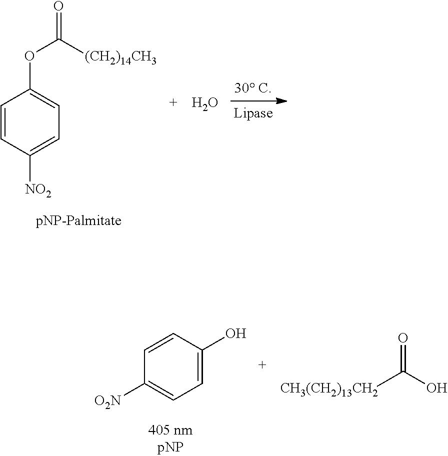

### Phospholipases:

The one or more lipolytic enzyme may include a polypeptide having phospholipase activity, preferably phospholipase A₁, phospholipase A₂, phospholipase B, phospholipase C, phospholipase D, lyso-phospholipases activity, and/or any combination thereof. In the process of the invention the one or more lipolytic enzyme may be a phospholipase, e.g., a single phospholipase such as A₁, A₂, B, C, or D; two or more phospholipases, e.g., two phospholipases, including, without limitation, both type A and B; both type A₁ and A₂; both type A₁ and B; both type A₂ and B; both type A₁ and C; both type A₂ and C; or two or more different phospholipases of the same type.

The one or more lipolytic enzyme may be a polypeptide having phospholipase activity, as well as having acyltransferase activity, e.g., a polypeptide selected from the polypeptides disclosed in WO 2003/100044, WO 2004/064537, WO 2005/066347, WO 2008/019069, WO 2009/002480, and WO 2009/081094. Acyltransferase activity may be e.g., determined by the assays described in WO 2004/064537.

The phospholipase may be selected from the polypeptides disclosed in WO 2008/036863 and WO 20003/2758. Suitable phospholipase preparations are PURIFINE® (available from Verenium) and LECITASE® ULTRA (available from Novozymes A/S). An enzyme having acyltransferase activity is available as the commercial enzyme preparation LYSOMAX® OIL (available from Danisco A/S).

Cutinases: The one or more lipolytic enzyme may include a polypeptide having cutinase activity.

The cutinase may e.g., be selected from the polypeptides disclosed in WO 2001/92502, in particular the Humicola insolens cutinase variants disclosed in Example 2.

Preferably, the one or more lipolytic enzyme is an enzyme having at least 60%, at least 70%, at least 80%, at least 90%, at least 95%, at least 96%, at least 97%, at least 98%, or even at least 99% identity to any of the aforementioned lipases, phospholipases, cutinases, and acyltransferases.

In one embodiment, the one or more lipolytic enzyme has at least 60%, at least 70%, at least 80%, at least 90%, at least 95%, at least 96%, at least 97%, at least 98%, at least or even at least 99% identity to the amino acid sequence shown as positions 1-269 of SEQ ID NO: 2 of WO 95/22615.

Enzyme sources and formulation: The one or more lipolytic enzyme used in the process of the invention may be derived or obtainable from any of the sources mentioned herein. The term “derived” means in this context that the enzyme may have been isolated from an organism where it is present natively, i.e. the identity of the amino acid sequence of the enzyme are identical to a native enzyme. The term “derived” also means that the enzymes may have been produced recombinantly in a host organism, the recombinant produced enzyme having either an identity identical to a native enzyme or having a modified amino acid sequence, e.g., having one or more amino acids which are deleted, inserted and/or substituted, i.e. a recombinantly produced enzyme which is a mutant and/or a fragment of a native amino acid sequence. Within the meaning of a native enzyme are included natural variants. Furthermore, the term “derived” includes enzymes produced synthetically by e.g., peptide synthesis. The term “derived” also encompasses enzymes which have been modified e.g., by glycosylation, phosphorylation etc., whether in vivo or in vitro. The term “obtainable” in this context means that the enzyme has an amino acid sequence identical to a native enzyme. The term encompasses an enzyme that has been isolated from an organism where it is present natively, or one in which it has been expressed recombinantly in the same type of organism or another, or enzymes produced synthetically by e.g., peptide synthesis. With respect to recombinantly produced enzyme the terms “obtainable” and “derived” refers to the identity of the enzyme and not the identity of the host organism in which it is produced recombinantly.

Accordingly, the one or more lipolytic enzyme may be obtained from a microorganism by use of any suitable technique. For instance, an enzyme preparation may be obtained by fermentation of a suitable microorganism and subsequent isolation of an enzyme preparation from the resulting fermented broth or microorganism by methods known in the art. The enzyme may also be obtained by use of recombinant DNA techniques. Such method normally comprises cultivation of a host cell transformed with a recombinant DNA vector comprising a DNA sequence encoding the enzyme in question and the DNA sequence being operationally linked with an appropriate expression signal such that it is capable of expressing the enzyme in a culture medium under conditions permitting the expression of the enzyme and recovering the enzyme from the culture. The DNA sequence may also be incorporated into the genome of the host cell. The DNA sequence may be of genomic, cDNA or synthetic origin or any combinations of these, and may be isolated or synthesized in accordance with methods known in the art.

The one or more lipolytic enzyme may be applied in any suitable formulation, e.g., as lyophilised powder or in aqueous solution.

### Sequence Identity

The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labelled “longest identity” (obtained using the—nobrief option) is used as the percent identity and is calculated as follows:

(Identical Residues×100)/(Length of Alignment−Total Number of Gaps in Alignment)

### Process Design

The present invention provides a process for reducing the level of free fatty acids in biodiesel/fatty acid alkyl esters, said process comprising
i) providing a composition comprising
   a. an oil phase/light phase that comprises fatty acid alkyl esters, free fatty acids and, optionally, a fatty acid feedstock; and
   b. a water phase/heavy phase that comprises alcohol and water;
ii) reducing the amount of water in said composition, such as reducing the water content of the water phase/heavy phase to be within the range of 0-15% by weight of the water phase/heavy phase and/or reducing the water content of the oil phase/light phase to be within the range of 200-600 ppm;
   and then reducing the amounts of free fatty acids, and optionally the amounts of said fatty acid feedstock in the oil phase/light phase by
iii) reacting said free fatty acids and/or said fatty acid feedstock with alcohol in the presence of one or more liquid lipolytic enzymes to produce fatty acid alkyl esters; and/or
iv) reacting the free fatty acids and/or said fatty acid feedstock with alcohol in the presence of one or more non-enzymatic catalysts to produce fatty acid alkyl esters.

In particular, the process according to the invention may comprise
i) providing a composition comprising
   a. an oil phase/light phase that comprises fatty acid alkyl esters, free fatty acids and, optionally, a fatty acid feedstock; and
   b. a water phase/heavy phase that comprises alcohol and water;
ii) reducing the amount of water in said composition, such as reducing the water content of the water phase/heavy phase to be within the range of 0-15% by weight of the water phase/heavy phase and/or reducing the water content of the oil phase/light phase to be within the range of 200-600 ppm;
iii) reducing the amounts of free fatty acids, and optionally the amounts of said fatty acid feedstock in the oil phase/light phase by reacting said free fatty acids and/or said fatty acid feedstock with alcohol in the presence of one or more liquid lipolytic enzymes to produce fatty acid alkyl esters;
and, optionally
iv) further reducing the amounts of free fatty acids and optionally the amounts of fatty acid feedstock in the oil phase/light phase by reacting the free fatty acids and/or said fatty acid feedstock with alcohol in the presence of one or more non-enzymatic catalysts to produce fatty acid alkyl esters.

The composition in i) may be provided by a reaction in which free fatty acids and/or a fatty acid feedstock is/are reacted with alcohol to produce fatty acid alkyl esters until the reaction has substantially reached equilibrium. In particular, “equilibrium may be defined as the point where there is no further net reduction of free fatty acids in the reaction mixture. Hence for the purpose of the present invention, the composition in i) may in particular be provided by a reaction, which has been allowed to proceed to a point where there is no further net reduction, or substantially no further net reduction of free fatty acids.

The composition in i) may in particular be provided by a reaction in which said fatty acid feedstock is reacted with alcohol in the presence of an amount of glycerol corresponding to 0 to 70% by weight of the water phase/heavy phase, an amount of water corresponding to 10 to 70.0% by weight of the water phase/heavy phase and an amount of alcohol, such as methanol, which is within the range of 10 to 50% by weight of the water phase/heavy phase.

In step ii), the water content of the water phase/heavy phase may be reduced to be within the range of 2-15% by weight of the water phase/heavy phase, such as to be within the range of 5-15% by weight of the water phase/heavy phase, such as to be within the range of 7-15% by weight of the water phase/heavy phase, such as to be within the range of 10-15% by weight of the water phase/heavy phase, such as to be within the range of 0-10% by weight of the water phase/heavy phase, such as to be within the range of 2-10% by weight of the water phase/heavy phase, such as to be within the range of 5-10% by weight of the water phase/heavy phase, such as to be within the range of 0-9% by weight of the water phase/heavy phase, such as to be within the range of 2-9% by weight of the water phase/heavy phase, or such as to be within the range of 5-9% by weight of the water phase/heavy phase.

The water content of the oil phase/light phase may also be reduced to be within the range of 200-600 ppm, such as within the range of 300-600 ppm, 400-600 ppm, 200-500 ppm, 200-400 ppm, or such as to be within the range of 300-500 ppm.

The glycerol content may correspond to 0 to 60% by weight of the water phase/heavy phase, such as to 0 to 50%, to 0 to 40%, to 0 to 30%, to 0 to 20%, to 2 to 60%, to 5 to 60%, to 10 to 60%, to 20 to 60%, to 30 to 60%, to 30 to 50%, to 5 to 50%, to 10 to 50%, to 20 to 50%, to 30 to 50%, to 2 to 40%, to 5 to 40%, to 10 to 40%, to 20 to 40%, to 2 to 30%m to 5 to 30%, or such as to 10 to 30% by weight of the water phase/heavy phase.

The water content may correspond to 10 to 60% by weight of the water phase/heavy phase, such as to 10 to 50%, to 10 to 40%, to 10 to 30%, to 10 to 20%, to 12 to 60%, to 15 to 60%, to 20 to 60%, to 30 to 60%, to 30 to 50%, 10 to 50%, to 20 to 50%, to 30 to 50%, to 10 to 40%, to 20 to 40%, or such as to 10 to 30% by weight of the water phase/heavy phase. As the skilled person will understand, the content of water in the water phase/heavy phase depends on the fatty acid feedstock used: if using mainly free fatty acids as substrate, the water phase/heavy phase will mainly comprise water whereas the use of a fatty acid feedstock with larger amounts of bound glycerol, will increase the amount of glycerol and decrease the amount of water in the water phase/heavy phase.

Preferably, the amount of alcohol, such as methanol, is within the range of 10 to 45% by weight of the water phase/heavy phase, such as within the range of 10 to 40%, 10 to 35%, 10 to 30%, 10 to 25%, 10 to 20%, 15 to 50%, 15 to 45%, 15 to 40%, 15 to 35%, 15 to 30%, 15 to 25%, 20 to 50%, 20 to 45%, 20 to 40%, 20 to 35%, 20 to 30%, 25 to 50%, 25 to 45%, 25 to 40%, or such as 25 to 35% by weight of the water phase/heavy phase.

In the process according to the invention, the composition in i) may be provided by a reaction, which comprises reacting free fatty acids and/or a fatty acid feed stock with alcohol until at least 90% (w/w) or such as at least 95% (w/w) of the fatty acid acyl groups or free fatty acids in said fatty acid feed stock have been converted to fatty acid alkyl esters.

In some embodiments of the invention, the fatty acids and/or said fatty acid feedstock in step iii) and/or in step iv) is/are reacted with alcohol until at least 80% (w/w), at least 85% (w/w), at least 90% (w/w) or such as at least 95% (w/w) of the free fatty acids and/or the fatty acid acyl groups in said fatty acid feed stock have been converted to fatty acid alkyl esters.

Preferably, the composition in step i) is provided by a reaction in which the one or more lipolytic enzymes is/are lipases. Preferred lipases are provided herein above.

The composition in i) may in particular be provided by a reaction in which the total amount of said one or more lipolytic enzymes is within the range of 0.005-5 g enzyme protein (EP)/kg oil phase/light phase or fatty acid feedstock, such as within the range of 0.005-2.5 g EP/kg oil phase/light phase or fatty acid feedstock, 0.005-1 g EP/kg oil phase/light phase or fatty acid feedstock, 0.005-0.75 g EP/kg oil phase/light phase or fatty acid feedstock, 0.005-0.5 g EP/kg oil phase/light phase or fatty acid feedstock, 0.005-0.25 g EP/kg oil phase/light phase or fatty acid feedstock, 0.005-0.1 g EP/kg oil phase/light phase or fatty acid feedstock, 0.005-0.075 g EP/kg oil phase/light phase or fatty acid feedstock, 0.005-0.05 g EP/kg oil phase/light phase or fatty acid feedstock, 0.005-0.025 g EP/kg oil phase/light phase or fatty acid feedstock, 0.005-0.01 g EP/kg oil phase/light phase or fatty acid feedstock, 0.01-5 g EP/kg oil phase/light phase or fatty acid feedstock, 0.02-5 g EP/kg oil phase/light phase or fatty acid feedstock, 0.03-5 g EP/kg oil phase/light phase or fatty acid feedstock, 0.04-5 g EP/kg oil phase/light phase or fatty acid feedstock, 0.05-5 g EP/kg oil phase/light phase or fatty acid feedstock, 0.06-5 g EP/kg oil phase/light phase or fatty acid feedstock, 0.07-5 g EP/kg oil phase/light phase or fatty acid feedstock, 0.08-5 g EP/kg oil phase/light phase or fatty acid feedstock, 0.09-5 g EP/kg oil phase/light phase or fatty acid feedstock, 0.1-5 g EP/kg oil phase/light phase or fatty acid feedstock, 0.2-5 g EP/kg oil phase/light phase or fatty acid feedstock, 0.3-5 g EP/kg oil phase/light phase or fatty acid feedstock, 0.4-5 g EP/kg oil phase/light phase or fatty acid feedstock, 0.5-5 g EP/kg oil phase/light phase or fatty acid feedstock, 0.6-5 g EP/kg oil phase/light phase or fatty acid feedstock, 0.7-5 g EP/kg oil phase/light phase or fatty acid feedstock, 0.8-5 g EP/kg oil phase/light phase or fatty acid feedstock, 0.9-5 g EP/kg oil phase/light phase or fatty acid feedstock, 1-5 g EP/kg oil phase/light phase or fatty acid feedstock, 2-5 g EP/kg oil phase/light phase or fatty acid feedstock, 3-5 g EP/kg oil phase/light phase or fatty acid feedstock, 4-5 g EP/kg oil phase/light phase or fatty acid feedstock, 0.01-4 g EP/kg oil phase/light phase or fatty acid feedstock, 0.02-3 g EP/kg oil phase/light phase or fatty acid feedstock, 0.03-2 g EP/kg oil phase/light phase or fatty acid feedstock, 0.04-1 g EP/kg oil phase/light phase or fatty acid feedstock, 0.05-0.9 g EP/kg oil phase/light phase or fatty acid feedstock, 0.06-0.8 g EP/kg oil phase/light phase or fatty acid feedstock, 0.07-0.7 g EP/kg oil phase/light phase or fatty acid feedstock, 0.08-0.6 g EP/kg oil phase/light phase or fatty acid feedstock, 0.09-0.5 g EP/kg oil phase/light phase or fatty acid feedstock, 0.1-0.4 g EP/kg oil phase/light phase or fatty acid feedstock, 0.1-0.3 g EP/kg oil phase/light phase or fatty acid feedstock, or such as within the range of 0.1-0.25 g EP/kg oil phase/light phase or fatty acid feedstock.

In step ii) of the process, the amount of water may be reduced by application of heat, such as by convection, conduction and/or radiation.

In particular, a gas stream may be used in step ii) to remove said water as humidity

In alternative embodiments, vacuum is used in step ii) to remove said water as humidity.

In currently preferred embodiments, the amount of water is reduced in step ii) by flash drying.

In the process according to the invention, the amount of alcohol in step iii) may correspond to 5-10% by weight of the oil phase/light phase, such as to 6 to 10%, 7 to 10%, 8 to 10%, 5 to 9%, 5 to 8%, or such as to 5 to 7% by weight of the oil phase/light phase.

The amount of alcohol in step iv) may correspond to 10-25% by weight of the oil phase/light phase, such as to 11 to 25%, 12 to 25%, 13 to 25%, 14 to 25%, 15 to 25%, 16 to 25%, 17 to 25%, 18 to 25%, 19 to 25%, 20 to 25%, 10 to 24%, 10 to 23%, 10 to 22%, 10 to 21%, 10 to 20%, 10 to 19%, 10 to 18%, 10 to 17%, 10 to 16%, 10 to 15%, or such as to 12 to 15% by weight of the oil phase/light phase.

In the process according to the invention, step iv) may comprise separating the water phase/heavy phase from the oil phase/light phase prior to further reducing the amounts of free fatty acids and optionally the amounts of fatty acid feedstock.

The duration of step iv) in the process according to the invention may be from 0.5-7 hours, such as 0.5-6 hours, 0.5-5 hours, 0.5-4 hours, 0.5-3 hours, 0.5-2 hours, 0.5-1 hour, 1-7 hours, 2-7 hours, 3-7 hours, or such as 4-7 hours.

The one or more lipolytic enzymes in step iii) may in particular be a lipase or one or more lipases, such as any one of the lipases disclosed herein before.

The total amount of said one or more lipolytic enzymes in step iii) may be within the range of 0.01-0.10 g enzyme protein (EP)/kg oil phase/light phase, or such as within the range of 0.02-0.10 g EP/kg oil phase/light phase, 0.03-0.01 g EP/kg oil phase/light phase, 0.04-0.10 g EP/kg oil phase/light phase, 0.05-0.1 g EP/kg oil phase/light phase, 0.06-0.1 g EP/kg oil phase/light phase or fatty acid feedstock, 0.07-0.1 g EP/kg oil phase/light phase, 0.08-0.01 g EP/kg oil phase/light phase, 0.01-0.09 g EP/kg oil phase/light phase, 0.01-0.08 g EP/kg oil phase/light phase, 0.01-0.07 g EP/kg oil phase/light phase, 0.01-0.06 g EP/kg oil phase/light phase, 0.01-0.05 g EP/kg oil phase/light phase, 0.01-0.04 g EP/kg oil phase/light phase, 0.01-0.03 g EP/kg oil phase/light phase, 0.02-0.08 g EP/kg oil phase/light phase or fatty acid feedstock, or such as 0.03-0.06 g EP/kg oil phase/light phase.

In the process according to the invention, the one or more non-enzymatic catalyst(s) used in step iv) may be selected from the group consisting of an acid catalyst, such as sulfonic acid, sulfuric acid, phosphoric acid, and hydrochloric acids, and a base catalyst, such as a metal alkoxide (e.g. sodium alkoxide or potassium alkoxide). In currently preferred embodiments, the catalyst is sulfonic acid.

In particular embodiments according to the invention, the one or more non-enzymatic catalyst(s) in step v) is/are immobilized on a solid resin, such as a macroporous, polymer-based resin. An example of such a resin, which is commercially available, is Lewatit GF 101 from Lanxess.

The reaction in step iv) may be conducted in a resin bed or column, in stirred reactor or in a continuous stirred reactor containing the one or more immobilized non-enzymatic catalysts. In particular the use of a continuous stirred reactor or a resin bed or column provides the advantage that the reaction may be conducted as a continuous reaction.

If choosing to run the reaction in a batch process, a conventional stirred reactor may be used. Alternatively, more complex reactors with integrated water removal systems, such as air bobbled batch reactors, may be employed. Such complex reactors would allow removal of water successively throughout the reaction.

One major advantage of the present invention, however, is the use of one main resin esterification step, without any complex reactor with integrated water removal system. Applying one or more non-enzymatic catalyst(s) in step iv) in a continuous stirred reactor or a resin bed or column as set forth in the present application, allows for a simple continuous process with no water removal, or at least with no substantial water removal, during the esterification process.

The solid resin is preferably packed in a resin bed or column having a height of at least one meter, such as at least 1.5 meters, at least 2 meters, at least 2.5 meters or such a height of as at least 3 meters. Particularly preferred columns have a height from 1-3 meters, such as from 1-2.5 meters, from 1-2 meters from 1-1.5 meters.

According to embodiments, in which the resin is packed in a resin bed or column, the holding time on the resin is preferably within the range of 1-4 hours, such as 1.5-4 hours, 1.5-3 hours, 2-3 hours or preferably in the range of 2.2-2.4 hours.

Step iv) may be performed in a pressurized system at a temperature within the range of 75-95° C., such as within the range of 80-95° C., 85-95° C., 90-95° C., 75-90° C., 75-85° C., 75-80° C., or such as within the range of 80-90° C.

The alcohol, which is used in the various steps in the process according to the invention is preferably a C1-C5 alcohol, more preferably ethanol or methanol.

The fatty acid feedstock used according to the present invention may be derived from one or more of algae oil, canola oil, coconut oil, castor oil, coconut oil, copra oil, corn oil, distiller's corn oil, cottonseed oil, flax oil, fish oil, grape seed oil, hemp oil, jatropha oil, jojoba oil, mustard oil, canola oil, palm oil, palm stearin, palm olein, palm kernel oil, peanut oil, rapeseed oil, rice bran oil, safflower oil, soybean oil, sunflower oil, tall oil, oil from halophytes, and/or animal fat, including tallow from pigs, beef and sheep, lard, chicken fat, fish oil, palm oil free fatty acid distillate, soy oil free fatty acid distillate, soap stock fatty acid material, yellow grease, and brown grease or any combination thereof.

In the process according to the invention, step iii) or iv) may be followed by a step in which soap/salts are formed from remaining free fatty acids in the oil phase/light phase by treatment with one or more alkaline agents, in the presence of said alcohol/said light phase.

The one or more alkaline agent may be added in an amount, which corresponds to 1.0-2.0 molar equivalents to the amount of free fatty acids, such as 1.2-2.0 molar equivalents, 1.3-2.0 molar equivalents, 1.4-2.0 molar equivalents, 1.5-2.0 molar equivalents, 1.6-2.0 molar equivalents, 1.7-2.0 molar equivalents, 1.8-2.0 molar equivalents, 1.0-0.9 molar equivalents, 1.0-0.8 molar equivalents, 1.0-0.7 molar equivalents, 1.0-0.6 molar equivalents, 1.0-0.5 molar equivalents, 1.0-0.4 molar equivalents, 1.0-0.3 molar equivalents, or such as 1.3-1.8 to molar equivalents to the amount of free fatty acids.

The treatment with one or more alkaline agents may comprise contacting the oil phase/light phase and optionally said water phase/hydrophilic phase with an alkaline agent or base selected from KOH or NaOH or a mixture thereof.

The treatment with one or more alkaline agents may preferably be performed at a temperature which is within the range of 35 to 70° C., such as within the range of 40 to 70° C., within the range of 45 to 70° C., within the range of 50 to 70° C., within the range of 55 to 70° C., or such within the range of 35 to 65° C.

The alkaline agent may in particular be sodium methoxide or potassium methoxide or a mixture of the two.

The process according to the invention may comprise a step of reducing the amounts of soap/fatty acid salts in the composition by subjecting the soap/fatty acid salts to acidification, such as by stoichiometric titration of the soap/fatty acid salts with acid, to produce free fatty acids, such as by contacting the soap/fatty acid salts with H₃PO₄ and/or H₂SO₄.

The said step of reducing the amounts of soap/fatty acid salts may in particular be performed prior to step iv).

The process according to the invention may further comprise separating the oil phase/light phase, containing the fatty acid alkyl esters from the hydrophilic phase/heavy phase.

As the skilled person will realize, the oil phase/light phase may be separated from the hydrophilic phase/heavy phase by gravity settling, decanting and/or centrifugation.

The process according to the invention may comprise drying said glycerol so as to remove e.g. water and alcohol, such as methanol or any other C1-C5 alcohol as disclosed herein, from the glycerol.

Preferably, the glycerol is purified, such as by drying and/or removal of alcohol to produce a composition, wherein the content of glycerol is above 95% (w/w), such as above 97% (w/w), above 97.5% (w/w), above 98% (w/w), above 98.5% (w/w), above 99% (w/w), above 99.5% (w/w), above 99.75% (w/w), above 99.8% (w/w) or is above 99.9% (w/w).

In particular, the glycerol may be subject to heat-vacuum distillation.

The process according to the invention may comprise subjecting the fatty acid alkyl esters to distillation, such as heat-vacuum distillation, wherein the fatty acid alkyl esters are evaporated and subsequently condensed.

In particular embodiments, the fatty acid alkyl esters are subject to heat-vacuum distillation at 240-260° C.

It is an advantage of the process provided according to the present invention that purification of the fatty acid alkyl esters, including any fatty alkyl esters produced in step iii) and/or iv), other than by distilling as set forth above is unnecessary and may be avoided. Nevertheless, if still desirable, the fatty acid alkyl esters may be subject to further purification.

The said purification may be performed by subjecting the fatty acid alkyl esters to water washing.

The purification may in particular be performed by allowing the fatty acid methyl esters to settle, such as by gravity settling, and then subjecting the settled fatty acid alkyl esters to water washing.

The process according to the invention may be a batch process, such as a process in which all of steps i), ii) and iii) or all of steps i), ii) and iv) are performed batch-wise. Alternatively, the process may be a semi-continuous, such as a process wherein one or more but not all of steps i), ii) and iii) are performed in a continuous manner, or wherein one or more but not all of steps i), ii) and iv) are performed in a continuous manner. Preferably, the process is a continuous process, wherein all of steps i), ii) and iii) or all of steps i), ii) and iv) are performed in a continuous manner.

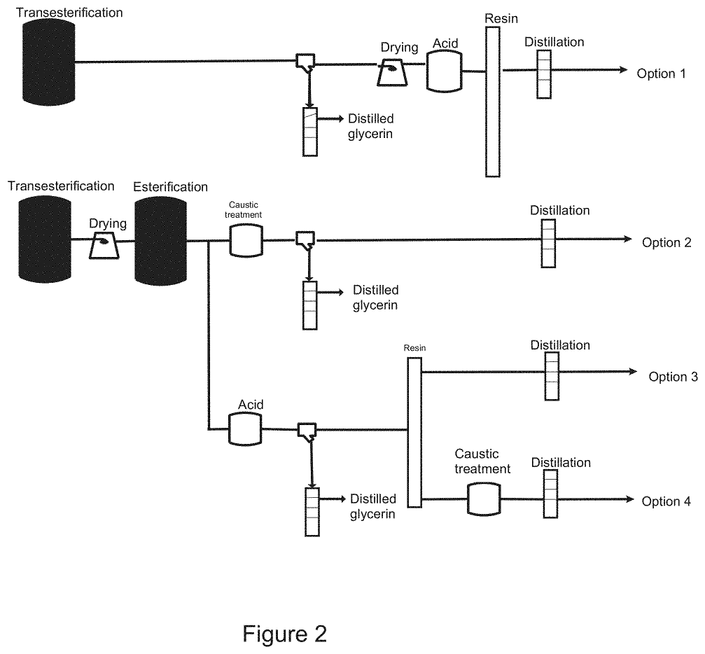

In specific embodiments according to the invention, which are illustrated as “option 1” in FIG. 2, the process according to the invention comprises
i) providing a composition comprising
   a. an oil phase/light phase that comprises fatty acid alkyl esters, free fatty acids and, optionally, a fatty acid feedstock; and
   b. a water phase/heavy phase that comprises alcohol and water;
ii) separating the oil phase/light phase, containing the fatty acid alkyl esters from the hydrophilic phase/heavy phase; and
iii) reducing the water content of the oil phase/light phase to be within the range of 200-600 ppm;
iv) optionally reducing the amounts of soap/fatty acid salts in the composition by subjecting the soap/fatty acid salts to acidification to produce free fatty acids, such as by contacting the soap/fatty acid salts with H₃PO₄ and/or H₂SO₄;
v) further reducing the amounts of free fatty acids and optionally the amounts of fatty acid feedstock in the oil phase/light phase by reacting the free fatty acids and/or said fatty acid feedstock with alcohol in the presence of one or more non-enzymatic catalysts to produce fatty acid alkyl esters; and
vi) purifying and/or distilling the fatty acid alkyl esters, including the fatty acid alkyl esters produced in step v).

In other specific embodiments according to the invention, which are illustrated as “option 2” in FIG. 2, the process comprises
i) providing a composition comprising
   a. an oil phase/light phase that comprises fatty acid alkyl esters, free fatty acids and, optionally, a fatty acid feedstock; and
   b. a water phase/heavy phase that comprises alcohol and water;
ii) reducing the amount of water in said composition, such as to be within the range of 0-15% by weight of the water phase/heavy phase;
iii) reducing the amounts of free fatty acids, and optionally the amounts of said fatty acid feedstock in the oil phase/light phase by reacting said free fatty acids and/or said fatty acid feedstock with alcohol and one or more liquid lipolytic enzymes to produce fatty acid alkyl esters;
iv) contacting the composition with one or more alkaline agents under conditions allowing formation of soap/salts from remaining free fatty acids in the oil phase/light phase;
v) separating the oil phase/light phase, containing the fatty acid alkyl esters from the hydrophilic phase/heavy phase; and
vi) purifying and/or distilling the fatty acid alkyl esters, including the fatty acid alkyl esters produced in step iii).

In still other specific embodiments according to the invention, which are illustrated as “option 3” in FIG. 2, the process comprises
i) providing a composition comprising
   a. an oil phase/light phase that comprises fatty acid alkyl esters, free fatty acids and, optionally, a fatty acid feedstock; and
   b. a water phase/heavy phase that comprises alcohol and water;
ii) reducing the amount of water in said composition, such as to be within the range of 0-15% by weight of the water phase/heavy phase;
iii) reducing the amounts of free fatty acids, and optionally the amounts of said fatty acid feedstock in the oil phase/light phase by reacting said free fatty acids and/or said fatty acid feedstock with alcohol and one or more liquid lipolytic enzymes to produce fatty acid alkyl esters;
iv) optionally reducing the amounts of soap/fatty acid salts in the composition by subjecting the soap/fatty acid salts to acidification to produce free fatty acids, such as by contacting the soap/fatty acid salts with H₃PO₄ and/or H₂SO₄.
v) separating the oil phase/light phase, containing the fatty acid alkyl esters from the hydrophilic phase/heavy phase;
vi) further reducing the amounts of free fatty acids and optionally the amounts of fatty acid feedstock in the oil phase/light phase by reacting the free fatty acids and/or said fatty acid feedstock with alcohol in the presence of one or more non-enzymatic catalysts to produce fatty acid alkyl esters; and
vii) purifying and/or distilling the fatty acid alkyl esters, including the fatty acid alkyl esters produced in steps iii) and vi).

In still further embodiments of the invention, which are illustrated as “option 4” in FIG. 2, the process comprises
i) providing a composition comprising
   a. an oil phase/light phase that comprises fatty acid alkyl esters, free fatty acids and, optionally, a fatty acid feedstock; and
   b. a water phase/heavy phase that comprises alcohol and water;
ii) reducing the amount of water in said composition, such as to be within the range of 0-15% by weight of the water phase/heavy phase;
iii) reducing the amounts of free fatty acids, and optionally the amounts of said fatty acid feedstock in the oil phase/light phase by reacting said free fatty acids and/or said fatty acid feedstock with alcohol and one or more liquid lipolytic enzymes to produce fatty acid alkyl esters;
iv) optionally reducing the amounts of soap/fatty acid salts in the composition by subjecting the soap/fatty acid salts to acidification to produce free fatty acids, such as by contacting the soap/fatty acid salts with H3PO4 and/or H2SO4;
v) separating the oil phase/light phase, containing the fatty acid alkyl esters from the hydrophilic phase/heavy phase;
vi) further reducing the amounts of free fatty acids and optionally the amounts of fatty acid feedstock in the oil phase/light phase by reacting the free fatty acids and/or said fatty acid feedstock with alcohol in the presence of one or more non-enzymatic catalysts to produce fatty acid alkyl esters;
vii) contacting the composition with one or more alkaline agents under conditions allowing formation of soap/salts from remaining free fatty acids in the oil phase/light phase; and
viii) purifying and/or distilling the fatty acid alkyl esters, including the fatty acid alkyl esters produced in steps iii) and vi).

The present invention is further described by the following examples that should not be construed as limiting the scope of the invention.

### EXAMPLES

### Example 1

### Hybrid Polishing Process, 1 L Scale

### Purpose:

The purpose was to test and develop a simple process with yields >97%, which can be implemented in large scale. The process developed herein is based on increasing conversion of FFA by an extra liquid enzyme step before caustic washing step.

### Procedure:

### 1. Mineral Acid Neutralization Step

a. 770 g dry RBD soybean oil was heated to 35 C/95 F and added 20 ppm NaOH as 1N solution≈0.385 g caustic solution. Mixing at 530 rpm for 15 minutes.
b. Water addition: total 2% water (including caustic solution)≈15.0 g water was added, mixing for 15 minutes.

### 2. Enzymatic Reaction.

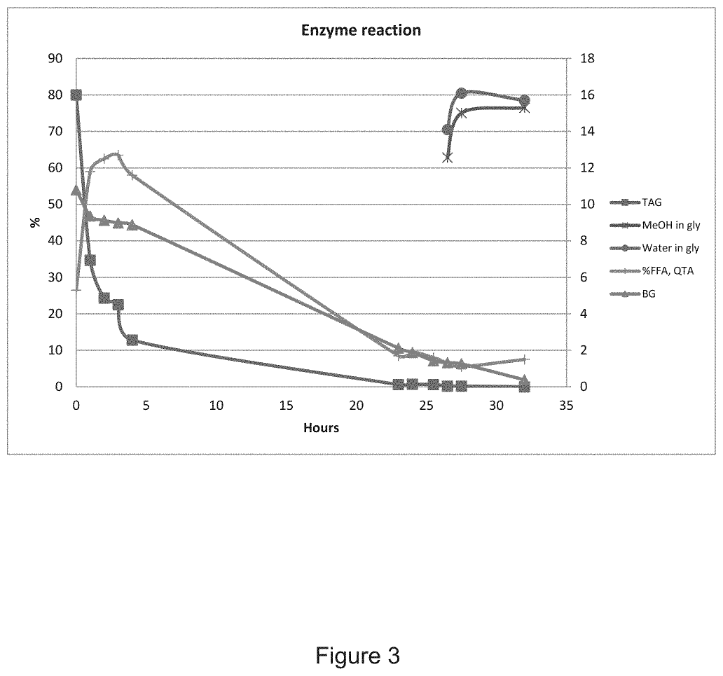

a. Lipase (lipase having the amino acid sequence set forth in SEQ ID NO: 2) addition was split in two dosings, 0.2% w/w at time 0 h and 0.1% addition at time 23 h≈1.54 ml and 0.77 ml respectively.
b. Total methanol dosing was 1.5 eqv starting with 21 ml added at time 0 h, 100 ml added continuously over first 20 hours and 14 ml added at 26.5 hours. Reaction ran for 32 hours with target of <0.5% BG. Final QTA readings: BG=0.38%, FFA (titration)=1.5%
   Changes in triacylglycerol, free fatty acids etc during interesterififcation are shown in FIG. 3.

### 3. Heating and Drying

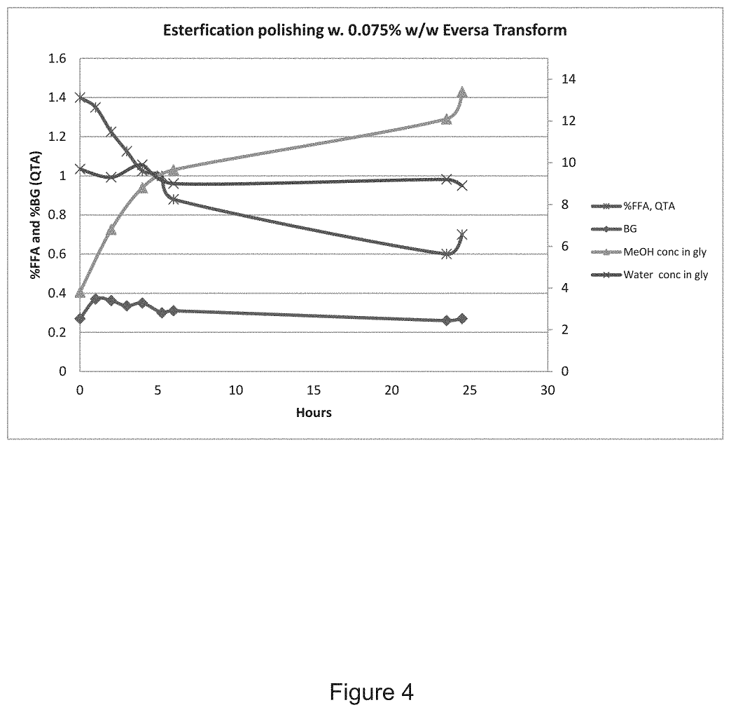

a. Reaction mixture was heated to 105 C/220 F for 1 hour, then cooled and dried at vacuum (end point 22 mbar) and 45 C/113 F for 1 hour. Heavy phase measurements (QTA): Water 15%, Methanol 3.3%, MONG 4.4%.
b. The mixture was heated to 80 C/180 F and dried (end point 20 mbar) for 1 hour. Heavy phase measurements (QTA): Water 9.9%, Methanol 0.1%, MONG 0.0%.
   4. Enzymatic Polishing Reaction w. Lipase (Lipase Having the Amino Acid Sequence Set Forth in SEQ ID NO: 2)
a. Dry mixture was cooled to 35 C/95 F and added 0.075% lipase≈0.50 ml and mixed at 530 rpm
b. Total methanol was 3.5% w/w. The dosing was split in 6 dosages: 10 ml at t=0 and t=1.5 h and 5 ml at 3 h, 5.5 h, 6.5 and 23.5 h. Reaction ran for 24.5 hours. End point FFA (titration)=0.8%
   QTA analysis data showing FFA reduction during enzymatic polishing are presented in FIG. 4.

### 5. Caustic Wash

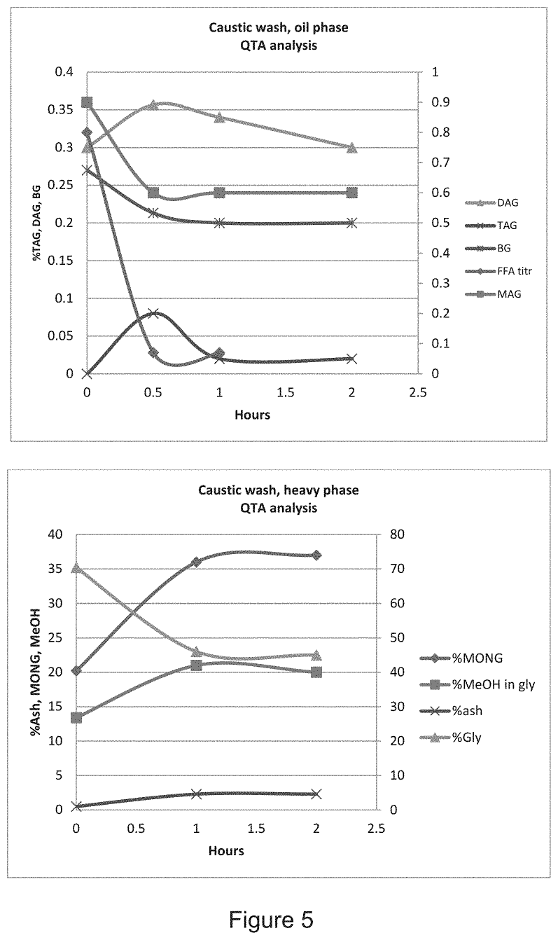

The mixture was heated to 60 C/140 F and added 1.5 eqv NaOH (relative to FFA content). The caustic was added as 3.2% w/w solution in methanol=62% conc. and water. Total addition was 40 g of caustic solution in 760 g mixture. Mixing at 530 for 2 hours. Final FFA (titration) <0.1% and BG=0.2%. QTA analyses of oil phase and heavy phase are shown in FIG. 5.

### 6. Settling, Washing and Drying

a. Settling by gravity for 30 min.
b. Static water washing by spraying with 4% v/v water followed by agitation for 30 min
c. Settling and decantation.
d. Drying of FAME phase at 100 C/212 F for 30 min. QTA measurements (B100):
   i. Acid number: 0.05
   ii. MAG: 0.4%
   iii. DAG: 0.27%
   iv. TAG: 0.01%
   v. BG: 0.19%
   vi. Total glycerin: 0.21%

### 7. Yield Loss Estimation by Acidulation

a. 10 g clear glycerol phase was added 0.73 ml 4N HCl and heated to 100 C for 30 min while shaken.
b. Centrifugation at 1500 rpm for 30 min. at 22 C.
c. Glycerol phase is sucked out by a pipette.
d. Weight of oil phase equal 0.7 g
e. Yield loss estimation:
   i. Total heavy phase 170 g
   ii. Oil amount in heavy phase: 0.7/10×170=11.9 g
   iii. Overall yield loss: 11.9 g oil relative to 770 g oil equals=1.5%

### 8. Yield Loss Estimation by QTA Measurement

a. Yield loss estimation:
   i. Total heavy phase 170 g
   ii. Total MONG content in heavy phase=11% MONG equal 11/100*170=18.7 g oil
   iii. Overall yield loss: 18.7 g oil relative to 770 g oil equals=2.4%

### Conclusions:

The above trial was successful in providing overall yields at 97-99%. Enzymatic polishing reaction with dried model substrate crude FAME from RBD soy bean oil, 0.075% Lipase (having the amino acid sequence set forth in SEQ ID NO: 2) and 3.5% methanol addition reduced FFA from 1.5% to 0.8% in 24 hours before final one pot caustic treatment.

### Example 2

### Resin Polishing on Crude Palm Oil

### Objective:

To study different resin polishing parameters such as column's height, methanol dosage, reaction temperature and FFA in the CPO FAME to achieve final FFA of <0.25%.

Part 1: Polishing with 0.4 m, 0.8 m and 1.2 m Resin Height on 1.3% Feed FFA
a) At 80 deg C with 15% of dried methanol and 0.4 meter column height, FFA could reduce from 1.3% to average 0.54%.
b) At 80 deg C with 20% of dried methanol and 0.8 meter column height, FFA could reduce from 1.3% to average 0.45%, whereas 1.2 meter column height is able to reduce FFA to an average of 0.37%.
c) At 90 deg C with 20% of dried methanol and 1.2 meter column height with flow of 4.3 bed volume/hr, FFA reduced from 1.3% to 0.24% (avg of 3 kg FAME going through the resin bed).
   Part 2: Polishing with 1.2 m Column Height on 2% Feed FFA
a) At 90 deg C with 20% of dried methanol and 1.2 meter column height, FFA able to reduce from 2% to average of 0.45% and further reduce down to 0.28-0.31% after 2^{nd} pass.
b) The targeted FFA could be achieving with higher column.

### Materials

Substrate: CPO FAME accumulated from ELN-14-PSSH-0008 (FFA-1.3%)
Resin: Lewatit GF 101 from Lanxess (CHT00077)
Chemical: Methanol dried (max 0.005% H2O) (Merck code: 1.06012.2500)

### Methodology

1. The resin was washed with 80 deg C deionized water to remove impurities before filling in to the 25 mm diameter×250 mm height glass column (total 0.4 meter resin height).
2. Dried methanol was pumped through the resin column at 90 deg C to remove the moisture in the resin down to 0.1% moisture before the actual polishing trial.
3. Mixture of 85% of CPO and 15% or 20% MeOH was incubated in 60 deg C water bath before pump through the resin.
4. The collected sample was measured for % moisture, before methanol evaporation for FFA analysis.
5. Further trials were conducted on several parameters such as methanol dosage (15% and 20%), temperature (80 deg C and 90 deg C), column height (0.4 m, 0.8 m and 1.2 m) and different FFA (1.3% and 2%) in the Feed FAME.

### Results and Discussion

Part 1: Polishing with 0.4 m, 0.8 m and 1.2 m Column Height on Feed FFA of 1.3%

TABLE 1
Results after resin polishing with 0.4 meter column height

| | | | Amount of | Ratio of | | |
|---|---|---|---|---|---|---|
| | Temperature of | % MeOH in | FAME + MeOH | (FAME + | | |
| Hour | column/deg C. | FAME | at outlet | MeOH):Resin | FFA % | % H2O |
| 1 | 80 | 16.7 | 174.96 | 0.87 | 0.44 | 0.227 |
| 2 | 80 | 16.7 | 150.09 | 0.75 | 0.43 | 0.202 |
| 3 | 90 | 16.7 | 140 | 0.7 | 0.47 | 0.228 |
| 4 | 80 | 16.7 | 124.75 | 0.62 | 0.56 | 0.352 |
| 5 | 90 | 16.7 | 114.49 | 0.57 | 0.55 | 0.328 |
| 6 | 90 | 20 | 87.74 | 0.44 | 0.63 | 0.289 |
| 7 | 90 | 20 | 96.31 | 0.48 | 0.68 | 0.501 |

Initially, the resin was packed in 2 glass columns with the dimension of 25 mm diameter×250 mm height column. This could be filled up to 20 g resin per column and the bed height is 20 cm per column. With this, 1.4 litre (1.1 kg) of dried methanol passing through the resin column, the moisture was reduced from 35.3% to 0.065% in 1 hr (Target: <0.1% moisture)

The CPO FAME with the FFA of 1.3% and moisture of 0.011% was blended as 85% with 15% of dried methanol as the feed for the polishing trial. Different settings were tried, such as the resin temperature, %methanol and bed volume flow to get the FFA reduced from 1.3% to 0.25%. However, the FFA could only reduced to 0.44% with the parameter as per Table 1. This suggested that the resin bed height must be at least 1 meter for a better conversion. height.

Therefore, the failure of the FFA reduction could be due to the insufficient height of the resin setup in the aquarium, hence the pre-treated resin from 2 big column (25 mm diameter×250 mm height) of 0.4 meter resin height was transferred to 4 and 6 small columns (10 mm diameter×250 mm height) of 0.8 meter and 1.2 meter height, respectively for better conversion.

TABLE 2
Results after resin polishing with 0.8
meter height (Aquarium at 80deg C.)

| Trial parameters | 1 | 2 | 3 |
|---|---|---|---|
| % CPO FAME | 85 | 85 | 85 |
| % MeOH | 15 | 15 | 15 |
| Number of columns | 4 × 10 g Resin | 4 × 10 g Resin | 4 × 10 g Resin |
| | (0.8 m) | (0.8 m) | (0.8 m) |
| Resin Temp/deg C. | 80 | 80 | 80 |
| Flow rate g/h | 141.24 g/h | 126.4 g/h | 60.8 g/h |
| Bed volume of | 3.5 | 3.2 | 1.5 |
| (FAME + MeOH) | | | |
| to resin | | | |
| Results | | | |
| % moisture before | 0.17 | 0.267 | 0.356 |
| evaporation | | | |
| % Initial FFA | 1.3 | 1.3 | 1.3 |
| % FFA | 0.36 | 0.47 | 0.51 |

TABLE 3
Results after resin polishing with 1.2 meter height (Aquarium at 80deg C.)

| | 4 | 5 | 6 | 7 | 8 | 9 | 10 |
|---|---|---|---|---|---|---|---|
| Trial parameters | | | | | | | |
| % CPO FAME | 80 | 80 | 80 | 80 | 80 | 80 | 80 |
| % MeOH | 20 | 20 | 20 | 20 | 20 | 20 | 20 |
| Number of | 6 × 10 g resin | 6 × 10 g resin | 6 × 10 g resin | 6 × 10 g resin | 6 × 10 g resin | 6 × 10 g resin | 6 × 10 g resin |
| columns | (1.2 m) | (1.2 m) | (1.2 m) | (1.2 m) | (1.2 m) | (1.2 m) | (1.2 m) |
| Resin Temp/deg C. | 80 | 80 | 80 | 80 | 80 | 80 | 80 |
| Flow rate g/h | 307.5 g/h | 400 g/h | 490 g/h | 250 g/h | 260 g/h | 210 g/h | — |
| Bed volume of | 5.1 | 6.6 | 8.2 | 4.2 | 4.3 | 3.5 | |
| (FAME + MeOH) | | | | | | | |
| to resin | | | | | | | |
| Results | | | | | | | |
| % moisture | 0.11 | 0.121 | 0.094 | 0.179 | 0.172 | 0.184 | — |
| before | | | | | | | |
| evaporation | | | | | | | |
| % Initial FFA | 1.3 | 1.3 | 1.3 | 0.78 | 1.3 | 1.3 | 1.3 |
| % FFA | 0.46 | 0.6 | 0.39 | 0.2 | 0.31 | 0.37 | 0.27 |

All of the trials with 0.8 meter and 1.2 meter column height were done 80 deg C. Referring to Table 2, FFA could only reduce to 0.36% with 0.8 meter column height, whereas Table 3 shows that 1.2 meter column height is able to reduce FFA from 1.3% to 0.22% using 80% CPO FAME and 20% Methanol as the feed. The ratio of FAME+Methanol to resin was approximately 4 bed volume.

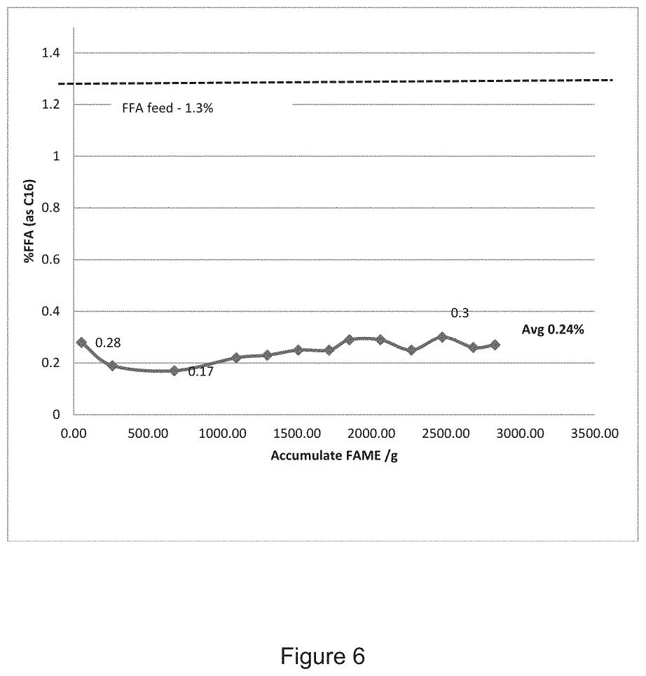

The Aquarium temperature was increased to the maximum of 90 deg C with 1.2 meter column height, 20% Methanol in the feed and flow of 4.3 bed volume/hr. As shown in FIG. 6, the FFA reduced from 1.3% to 0.24% (avg of 3 kg FAME going through the resin bed). The FFA after resin treatment ranging from (0.17-0.30)%. Feed of FAME sample was dried before added with dried methanol. The moisture content was about 0.02%.

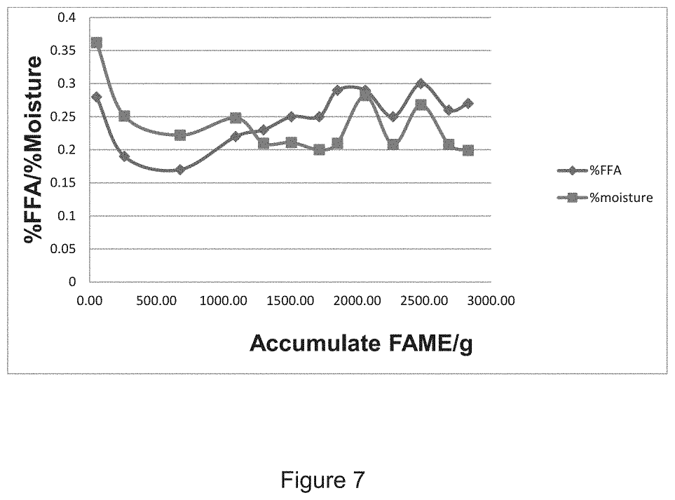

Refer to FIG. 7, moisture of FAME after resin treatment is 0.24% in average. The moisture was tested after the resin treatment, before methanol/moisture evaporation for FFA analysis. By stoichiometric calculation, 1% palmitic acid FFA converting to FAME will generate 0.07% moisture. The higher moisture content measured could be due to the moisture absorption by biodiesel at ambient environment.

Part 2: Polishing with 1.2 m Resin Height on 2% Feed FFA

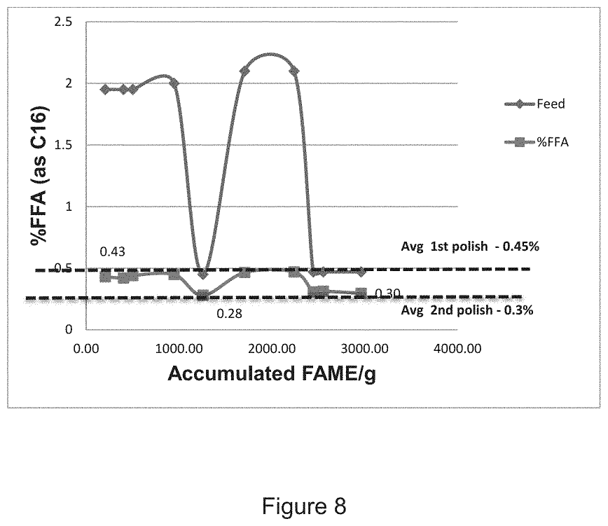

With the same resin height of 1.2 meter and aquarium temperature of 90 deg C, higher feed FFA of 2% and methanol (80%:20%) in the feed were tested. As shown in FIG. 8, the system was able to reduce FFA from 2% to 0.45% (Avg) after first round of resin polishing. The FAME collected from the first round of polishing which consisted of 18-19% methanol was passed the resin for second time. Then, the system was able to reduce FFA from 0.45% (avg) to 0.28-0.31%.

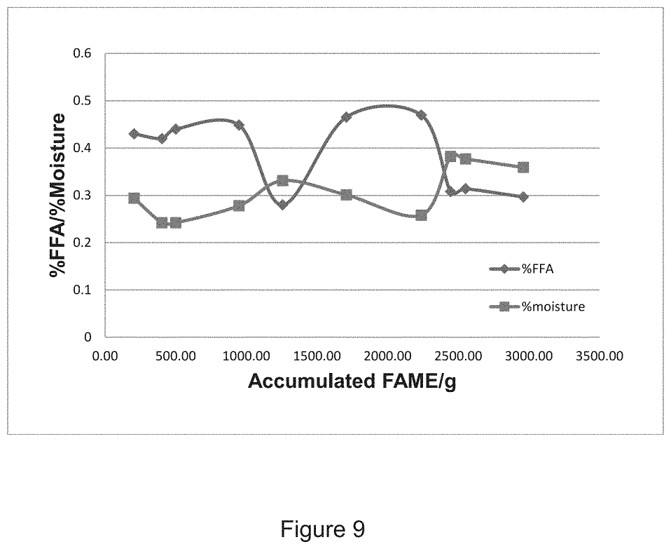

FIG. 9 shows that when FFA reduced from 2% to 0.45%, the moisture in the FAME is 0.27% (avg). Further FFA reduction from 0.45% to 0.3% causes the moisture to increase to 0.36% (avg). The moisture increment in the collected sample might due to water absorption from the humidity.

Conclusion

TABLE 4
Short summary overview on FFA polishing

| Resin Height/meter | 0.4 meter | 0.8 meter | 1.2 meter | 1.2 meter | 1.2 meter |
|---|---|---|---|---|---|
| % CPO FAME | 85 | 80 | 80 | 80 | 80 |
| % MeOH | 15 | 20 | 20 | 20 | 20 |
| Temperature for | 80 & 90 | 80 | 80 | 90 | 90 |
| Feed/deg C. | | | | | |

| | | | | | First | |
|---|---|---|---|---|---|---|
| Results | | | | | polishing | Second polishing |
| % Initial FFA | 1.3 | 1.3 | 1.3 | 1.3 | 2 | 0.45 |
| % FFA (average) | 0.54 | 0.45 | 0.37 | 0.24 | 0.45 | 0.3 |

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A process for reducing the level of free fatty acids in biodiesel/fatty acid alkyl esters, said process comprising
i) providing a composition comprising
a. an oil phase/light phase that comprises fatty acid alkyl esters, free fatty acids and, optionally, a fatty acid feedstock; and
b. a water phase/heavy phase that comprises alcohol and water;
ii) reducing the amount of water in said composition, such as reducing the water content of the water phase/heavy phase to be within the range of 0-15% by weight of the water phase/heavy phase and/or reducing the water content of the oil phase/light phase to be within the range of 200-600 ppm;
and then reducing the amounts of free fatty acids, and optionally the amounts of said fatty acid feedstock in the oil phase/light phase by
iii) reacting said free fatty acids and/or said fatty acid feedstock with alcohol in the presence of one or more liquid lipolytic enzymes to produce fatty acid alkyl esters; and/or
iv) reacting the free fatty acids and/or said fatty acid feedstock with alcohol in the presence of one or more non-enzymatic catalysts to produce fatty acid alkyl esters.

2. The process according to claim 1, said process comprising
i) providing a composition comprising
a. an oil phase/light phase that comprises fatty acid alkyl esters, free fatty acids and, optionally, a fatty acid feedstock; and
b. a water phase/heavy phase that comprises alcohol and water;
ii) reducing the amount of water in said composition, such as reducing the water content of the water phase/heavy phase to be within the range of 0-15% by weight of the water phase/heavy phase and/or reducing the water content of the oil phase/light phase to be within the range of 200-600 ppm;
iii) reducing the amounts of free fatty acids, and optionally the amounts of said fatty acid feedstock in the oil phase/light phase by reacting said free fatty acids and/or said fatty acid feedstock with alcohol in the presence of one or more liquid lipolytic enzymes to produce fatty acid alkyl esters;
and, optionally
iv) further reducing the amounts of free fatty acids and optionally the amounts of fatty acid feedstock in the oil phase/light phase by reacting the free fatty acids and/or said fatty acid feedstock with alcohol in the presence of one or more non-enzymatic catalysts to produce fatty acid alkyl esters.

3. The process according to claim 1, wherein the composition in i) is provided by a reaction in which free fatty acids and/or a fatty acid feedstock is/are reacted with alcohol to produce fatty acid alkyl esters until the reaction has substantially reached equilibrium, such as until the reaction proceeds with substantially no further net reduction of free fatty acids.

4. The process according to claim 1, wherein the composition in i) is provided by a reaction in which said fatty acid feedstock is reacted with alcohol in the presence of an amount of glycerol corresponding to 0 to 70% by weight of the water phase/heavy phase, an amount of water corresponding to 10 to 70.0% by weight of the water phase/heavy phase and an amount of alcohol, such as methanol, which is within the range of 10 to 50% by weight of the water phase/heavy phase.

5. The process according to claim 1, wherein the composition in i) is provided by a reaction which comprises reacting free fatty acids and/or a fatty acid feed stock with alcohol until at least 90% (w/w) or such as at least 95% (w/w) of the fatty acid acyl groups or free fatty acids in said fatty acid feed stock have been converted to fatty acid alkyl esters.

6. (canceled)

7. The process according to claim 1, wherein the amount of water in the composition provided in step i) is in the range of 10-70% by weight of the water phase/heavy phase.

8. The process according to claim 1, wherein the composition in i) is provided by a reaction in which the one or more lipolytic enzymes is/are lipases.

9. The process according to claim 1, wherein the composition in i) is provided by a reaction in which the total amount of said one or more lipolytic enzymes is within the range of 0.005-5 g enzyme protein (EP)/kg oil or fatty acid feedstock.

10-13. (canceled)

14. The process according to claim 1, wherein the amount of alcohol in step iii) corresponds to 5-10% by weight of the light phase.

15. The process according to any of the claims claim 1, wherein the amount of alcohol in step iv) corresponds to 10-25% by weight of the light phase.

16-17. (canceled)

18. The process according to claim 1, wherein the one or more lipolytic enzymes in step ii) is/are lipases.

19. The process according to claim 1, wherein the total amount of said one or more lipolytic enzymes in step iii) is within the range of 0.01-0.10 g enzyme protein (EP)/kg oil.

20. The process according to claim 1 wherein the one or more non-enzymatic catalyst(s) in step iv) is/are selected from the group consisting of an acid catalyst, such as sulfonic acid, sulfuric acid, phosphoric acid, and hydrochloric acids, and a base catalyst, such as a metal alkoxide (e.g. sodium alkoxide or potassium alkoxide).

21-24. (canceled)

25. The process according to claim 1, wherein the fatty acid feedstock is derived from one or more of algae oil, canola oil, coconut oil, castor oil, coconut oil, copra oil, corn oil, distiller's corn oil, cottonseed oil, flax oil, fish oil, grape seed oil, hemp oil, jatropha oil, jojoba oil, mustard oil, canola oil, palm oil, palm stearin, palm olein, palm kernel oil, peanut oil, rapeseed oil, rice bran oil, safflower oil, soybean oil, sunflower oil, tall oil, oil from halophytes, and/or animal fat, including tallow from pigs, beef and sheep, lard, chicken fat, fish oil, palm oil free fatty acid distillate, soy oil free fatty acid distillate, soap stock fatty acid material, yellow grease, and brown grease or any combination thereof.

26. The process according to claim 1, wherein step iii) or iv) is followed by a step in which soap/salts are formed from remaining free fatty acids in the oil phase/light phase by treatment with one or more alkaline agents, in the presence of said alcohol/said light phase.

37-30. (canceled)

31. The process according to claim 1, further comprising a step of reducing the amounts of soap/fatty acid salts in the composition by subjecting the soap/fatty acid salts to acidification, such as by stoichiometric titration of the soap/fatty acid salts with acid, to produce free fatty acids, such as by contacting the soap/fatty acid salts with H₃PO₄ and/or H₂SO₄.

33-34. (canceled)

35. The process according to claim 4, comprising drying said glycerol so as to remove e.g. water and alcohol, such as methanol, from the glycerol.

36-37. (canceled)

38. The process according to claim 1, comprising subjecting the fatty acid alkyl esters to distillation, such as heat-vacuum distillation, wherein the fatty acid alkyl esters are evaporated and subsequently condensed.

39-42. (canceled)

43. The process according to claim 1, said process comprising
i) providing a composition comprising
a. an oil phase/light phase that comprises fatty acid alkyl esters, free fatty acids and, optionally, a fatty acid feedstock; and
b. a water phase/heavy phase that comprises alcohol and water;
ii) separating the oil phase/light phase, containing the fatty acid alkyl esters from the hydrophilic phase/heavy phase; and
iii) reducing the water content of the oil phase/light phase to be within the range of 200-600 ppm;
iv) optionally reducing the amounts of soap/fatty acid salts in the composition by subjecting the soap/fatty acid salts to acidification to produce free fatty acids, such as by contacting the soap/fatty acid salts with H₃PO₄ and/or H₂SO₄;
v) further reducing the amounts of free fatty acids and optionally the amounts of fatty acid feedstock in the oil phase/light phase by reacting the free fatty acids and/or said fatty acid feedstock with alcohol in the presence of one or more non-enzymatic catalysts to produce fatty acid alkyl esters; and
vi) purifying and/or distilling the fatty acid alkyl esters, including the fatty acid alkyl esters produced in step v).

44. The process according to claim 1, said process comprising
i) providing a composition comprising
a. an oil phase/light phase that comprises fatty acid alkyl esters, free fatty acids and, optionally, a fatty acid feedstock; and
b. a water phase/heavy phase that comprises alcohol and water;
ii) reducing the amount of water in said composition, such as to be within the range of 0-15% by weight of the water phase/heavy phase;
iii) reducing the amounts of free fatty acids, and optionally the amounts of said fatty acid feedstock in the oil phase/light phase by reacting said free fatty acids and/or said fatty acid feedstock with alcohol and one or more liquid lipolytic enzymes to produce fatty acid alkyl esters;
iv) contacting the composition with one or more alkaline agents under conditions allowing formation of soap/salts from remaining free fatty acids in the oil phase/light phase;
v) separating the oil phase/light phase, containing the fatty acid alkyl esters from the hydrophilic phase/heavy phase; and
vi) purifying and/or distilling the fatty acid alkyl esters, including the fatty acid alkyl esters produced in step iii).

45. The process according to claim 1, said process comprising
i) providing a composition comprising
a. an oil phase/light phase that comprises fatty acid alkyl esters, free fatty acids and, optionally, a fatty acid feedstock; and
b. a water phase/heavy phase that comprises alcohol and water;
ii) reducing the amount of water in said composition, such as to be within the range of 0-15% by weight of the water phase/heavy phase;
iii) reducing the amounts of free fatty acids, and optionally the amounts of said fatty acid feedstock in the oil phase/light phase by reacting said free fatty acids and/or said fatty acid feedstock with alcohol and one or more liquid lipolytic enzymes to produce fatty acid alkyl esters;
iv) optionally reducing the amounts of soap/fatty acid salts in the composition by subjecting the soap/fatty acid salts to acidification to produce free fatty acids, such as by contacting the soap/fatty acid salts with H₃PO₄ and/or H₂SO₄.
v) separating the oil phase/light phase, containing the fatty acid alkyl esters from the hydrophilic phase/heavy phase;
vi) further reducing the amounts of free fatty acids and optionally the amounts of fatty acid feedstock in the oil phase/light phase by reacting the free fatty acids and/or said fatty acid feedstock with alcohol in the presence of one or more non-enzymatic catalysts to produce fatty acid alkyl esters
vii) purifying and/or distilling the fatty acid alkyl esters, including the fatty acid alkyl esters produced in steps iii) and vi).

46. The process according to claim 1, said process comprising
i) providing a composition comprising
a. an oil phase/light phase that comprises fatty acid alkyl esters, free fatty acids and, optionally, a fatty acid feedstock; and
b. a water phase/heavy phase that comprises alcohol and water;
ii) reducing the amount of water in said composition, such as to be within the range of 0-15% by weight of the water phase/heavy phase;
iii) reducing the amounts of free fatty acids, and optionally the amounts of said fatty acid feedstock in the oil phase/light phase by reacting said free fatty acids and/or said fatty acid feedstock with alcohol and one or more liquid lipolytic enzymes to produce fatty acid alkyl esters;
iv) optionally reducing the amounts of soap/fatty acid salts in the composition by subjecting the soap/fatty acid salts to acidification to produce free fatty acids, such as by contacting the soap/fatty acid salts with H3PO4 and/or H2SO4;
v) separating the oil phase/light phase, containing the fatty acid alkyl esters from the hydrophilic phase/heavy phase;
vi) further reducing the amounts of free fatty acids and optionally the amounts of fatty acid feedstock in the oil phase/light phase by reacting the free fatty acids and/or said fatty acid feedstock with alcohol in the presence of one or more non-enzymatic catalysts to produce fatty acid alkyl esters;
vii) contacting the composition with one or more alkaline agents under conditions allowing formation of soap/salts from remaining free fatty acids in the oil phase/light phase; and
viii) purifying and/or distilling the fatty acid alkyl esters, including the fatty acid alkyl esters produced in steps iii) and vi).
